SEQUENCE LISTING (8 sequences)

SEQ ID NO: 1 - DNA, 1052 bp, Ashbya gossypii
  CDS (137)..(1042)
aaaggcttttccgtaggtgctttgtcattcaacaatccacgtcggaattggcgactatatagtgtagggcccataaagcagtagtcggtgttgatagctgtgtcagaccaactctttgttaattactgaagctgatatgactgaatacacagtgccagaagtgacctgtgtcgcacgcgcgcgcataccgacggtacagggcaccgatgtcttcctccatctataccacaactcgatcgacagcaaggaacacctagcgattgtcttcggcgagaacatacgctcgcggagtctgttccggtaccggaaagacgacacgcagcaggcgcggatggtgcggggcgcctacgtgggccagctgtaccccgggcggaccgaggcagacgcggatcggcgtcagggcctggagctgcggtttgatgagacagggcagctggtggtggagcgggcgacgacgtggaccagggagccgacactggtgcggctgcactcggagtgttacacgggcgagacggcgtggagcgcgcggtgcgactgcggggagcagttcgaccaggcgggtaagctgatggctgcggcgacagagggcgaggtggttggcggtgcggggcacggcgtgatcgtgtacctgcggcaggagggccgcggcatcgggctaggcgagaagctgaaggcgtacaacctgcaggacctgggcgcggacacggtgcaggcgaacgagctgctcaaccaccctgcggacgcgcgcgacttctcgttggggcgcgcaatcctactggacctcggtatcgaggacatccggttgctcacgaataaccccgacaaggtgcagcaggtgcactgtccgccggcgctacgctgcatcgagcgggtgcccatggtgccgctttcatggactcagcccacacagggcgtgcgctcgcgcgagctggacggctacctgcgcgccaaggtcgagcgcatggggcacatgctgcagcggccgctggtgctgcacacgtctgcggcggccgagctcccccgcgccaacacacacatataatctttgctat

SEQ ID NO: 2 - protein, 301 aa, Ashbya gossypii
MTEYTVPEVTCVARARIPTVQGTDVFLHLYHNSIDSKEHLAIVFGENIRSRSLFRYRKDDTQQARMVRGAYVGQLYPGRTEADADRRQGLELRFDETGQLVVERATTWTREPTLVRLHSECYTGETAWSARCDCGEQFDQAGKLMAAATEGEVVGGAGHGVIVYLRQEGRGIGLGEKLKAYNLQDLGADTVQANELLNHPADARDFSLGRAILLDLGIEDIRLLTNNPDKVQQVHCPPALRCIERVPMVPLSWTQPTQGVRSRELDGYLRAKVERMGHMLQRPLVLHTSAAAELPRANTHI

SEQ ID NO: 3 - DNA, 2050 bp, Ashbya gossypii
  CDS (209)..(2038)
agacgtcacagatatactactgatgttgttctccagagtatactacgcccctaccatattcgatcttgtggtattgacgatattcctctgtttggttttactggcactattccgtttgacggtatagcgctattcgttcatagtgacacatgcggcactagctattcagcgaatcctttataaactgctacttaacgttcgtaacaccatgctcaaaggcgttcctggccttctttttaaggagacgcaacgtcatctgaaacccaggctggttaggattatggaaaacacatcgcaggatgagagtcgcaaaagacaggtcgcttcgaacttgagcagcgatgccgatgagggctcgccggcagttacgaggccggttaaaatcaccaaacgcctcaggaagaagaacctcgggacaggcgagctacgggacaaagcaggattcaagttgaaggtgcaagacgtgagcaaaaaccgtcacagacaggtcgatccggaatacgaagtcgtggtagatggcccgatgcgcaagatcaaaccgtatttcttcacatacaagactttctgcaaggagcgctggagagatcggaagttgcttgatgtgtttgtggatgaatttcgggaccgcgataggccttactacgagaaagtcatcggttcgggtggtgtgctcctgaacggtaagtcatcgacgttagatagcgtattgcgtaatggagacctcatttcgcacgagctgcaccgtcatgagccaccggtctcctctaggccgattaggacggtgtacgaagatgatgacatcctggtgattgacaagcccagcgggattccagcccatcccaccgggcgttaccgcttcaactccattacgaaaatacttgaaaaacagcttggatacactgttcatccatgtaaccgactggaccgcctaaccagtggcctaatgttcttggcaaaaactccaaagggagccgatgagatgggtgatcagatgaaggcgcgcgaagtgaagaaagaatatgttgcccgggttgttggggaatttcctataggtgagatagttgtggatatgccactgaagactatagagccgaagcttgccctaaacatggtttgcgacccggaagacgaagcgggcaagggcgctaagacgcagttcaaaagaatcagctacgatggacaaacgagcatagtcaagtgccaaccgtacacgggccggacgcatcagatccgtgttcacttgcaatacctgggcttcccaattgccaacgatccgatttattccaatccgcacatatggggcccaagtctgggcaaggaatgcaaagcagactacaaggaggtcatccaaaaactaaacgaaattggtaagactaaatctgcggaaagttggtaccattctgattcccaaggtgaagttttgaaaggggaacaatgcgatgaatgtggcaccgaactgtacactgacccgggcccgaatgatcttgacttatggttgcatgcatatcggtatgaatccactgaactggatgagaacggtgctaaaaagtggagttactctactgcgtttcctgagtgggctcttgagcagcacggcgacttcatgcggcttgccatcgaacaggctaagaaatgcccacccgcgaagacatcatttagcgttggtgccgtgttagttaatgggaccgagattttggccactggttactcacgggagctggaaggcaacacgcacgctgaacaatgtgcacttcaaaaatattttgaacaacataaaaccgacaaggttcctattggtacagtaatatacacgactatggagccttgttctctccgtctcagtggtaataaaccgtgtgttgagcgtataatctgccagcagggtaatattactgctgtttttgttggcgtacttgagccagacaacttcgtgaagaacaatacaagtcgtgcgctattggaacaacatggtatagactatattcttgtccctgggtttcaagaagaatgtactgaagccgcattgaagggtcattgattttgctgcgaa

SEQ ID NO: 4 - protein, 609 aa, Ashbya gossypii
MLKGVPGLLFKETQRHLKPRLVRIMENTSQDESRKRQVASNLSSDADEGSPAVTRPVKITKRLRKKNLGTGELRDKAGFKLKVQDVSKNRHRQVDPEYEVVVDGPMRKIKPYFFTYKTFCKERWRDRKLLDVFVDEFRDRDRPYYEKVIGSGGVLLNGKSSTLDSVLRNGDLISHELHRHEPPVSSRPIRTVYEDDDILVIDKPSGIPAHPTGRYRFNSITKILEKQLGYTVHPCNRLDRLTSGLMFLAKTPKGADEMGDQMKAREVKKEYVARVVGEFPIGEIVVDMPLKTIEPKLALNMVCDPEDEAGKGAKTQFKRISYDGQTSIVKCQPYTGRTHQIRVHLQYLGFPIANDPIYSNPHIWGPSLGKECKADYKEVIQKLNEIGKTKSAESWYHSDSQGEVLKGEQCDECGTELYTDPGPNDLDLWLHAYRYESTELDENGAKKWSYSTAFPEWALEQHGDFMRLAIEQAKKCPPAKTSFSVGAVLVNGTEILATGYSRELEGNTHAEQCALQKYFEQHKTDKVPIGTVIYTTMEPCSLRLSGNKPCVERIICQQGNITAVFVGVLEPDNFVKNNTSRALLEQHGIDYILVPGFQEECTEAALKGH

SEQ ID NO: 5 - DNA, 730 bp, Ashbya gossypii
  CDS (195)..(713)
ttgagctatatgtaagtctattaattgattactaatagcaatttatggtatcctctgttctgcatatcgacggttactcacgtgatgatcagcttgaggcttcgcggataaagttccatcgattactataaaaccatcacattaaacgttcactataggcatacacacagactaagttcaagttagcagtgacaatgattaagggattaggcgaagttgatcaaacctacgatgcgagctctgtcaaggttggcattgtccacgcgagatggaacaagactgtcattgacgctctcgtccaaggtgcaattgagaaactgcttgctatgggagtgaaggagaagaatatcactgtaagcaccgttccaggtgcgtttgaactaccatttggcactcagcggtttgccgagctgaccaaggcaagtggcaagcatttggacgtggtcatcccaattggagtcctgatcaaaggcgactcaatgcactttgaatatatatcagactctgtgactcatgccttaatgaacctacagaagaagattcgtcttcctgtcatttttggtttgctaacgtgtctaacagaggaacaagcgttgacacgtgcaggcctcggtgaatctgaaggcaagcacaaccacggtgaagactggggtgctgctgccgtggagatggctgtaaagtttggcccacgcgccgaacaaatgaagaagtgaatattaaaaaatcacta

SEQ ID NO: 6 - protein, 172 aa, Ashbya gossypii
MIKGLGEVDQTYDASSVKVGIVHARWNKTVIDALVQGAIEKLLAMGVKEKNITVSTVPGAFELPFGTQRFAELTKASGKHLDVVIPIGVLIKGDSMHFEYISDSVTHALMNLQKKIRLPVIFGLLTCLTEEQALTRAGLGESEGKHNHGEDWGAAAVEMAVKFGPRAEQMKK

SEQ ID NO: 7 - DNA, 1109 bp, Ashbya gossypii
  CDS (352)..(1092)
gaagaagcgcaggcgccagtccgagctggaggagaacgaggcggcgcggttgacgaacagcgcgctgcccatggacgatgcgggtatacagacggcgggtatacagacggcgggtggtgccgagagaggcaccaggccggcttcctccagcgatgcaaggaagagaaggggaccagaggcgaagttcaagccatctaaggtacagaagccccaattgaagcgaactgcatcgtcccgggcggatgagaacgagttctcgatattatagaggcccccgtttcgagtgattggcgtcaaaaacggctatctgccttcgtccgcccccaccaccctcgggaacactggcaaaccatggcgctaataccactttctcaagatctggctgatatactagcaccgtacttaccgacaccaccggactcatccgcacgcctgccgtttgtcacgctgacgtatgcgcagtccctagatgctcgtatcgcgaagcaaaagggtgaaaggacggttatttcgcatgaggagaccaagacaatgacgcattatctacgctaccatcatagcggcatcctgattggctcgggcacagcccttgcggacgacccggatctcaattgccggtggacacctgcagcggacggggcggattgcaccgaacagtcttcaccacgacccattatcttggatgttcggggcagatggagataccgcgggtccaaaatagagtatctgcataaccttggcaaggggaaggcgcccatagtggtcacggggggtgagccggaggtccgcgaactaggcgtcagttacctgcagctgggtgtcgacgagggtggccgcttgaattggggcgagttgtttgagcgactctattctgagcaccacctggaaagtgtcatggtcgaaggcggcgcggaggtgctcaaccagctgctgctgcgcccagatattgtggacagtctggtgatcacgataggatccaagttcctgggctcactaggtgttgcggtctcaccagctgaggaggtgaacctagagcatgtgaactggtggcacggaacaagtgacagtgttttgtgcggccggctcgcatagcggttatgactggtcta

SEQ ID NO: 8 - protein, 246 aa, Ashbya gossypii
MALIPLSQDLADILAPYLPTPPDSSARLPFVTLTYAQSLDARIAKQKGERTVISHEETKTMTHYLRYHHSGILIGSGTALADDPDLNCRWTPAADGADCTEQSSPRPIILDVRGRWRYRGSKIEYLHNLGKGKAPIVVTGGEPEVRELGVSYLQLGVDEGGRLNWGELFERLYSEHHLESVMVEGGAEVLNQLLLRPDIVDSLVITIGSKFLGSLGVAVSPAEEVNLEHVNWWHGTSDSVLCGRLA

(19) United States
(12) Patent Application Publication
(10) Pub. No.: US 2005/0239161 A1
(43) Pub. Date: Oct. 27, 2005

(54) Genetic strain optimization for improving the production of riboflavin

(76) Inventors: Henning Althofer, Wachenheim (DE); Jose L. Revuelta Doval, Salamanca (ES)

Correspondence Address: Morrison & Foerster, Suite 300, 1650 Tysons Boulevard, McLean, VA, 22102-3915, US

(21) Appl. No.: 10/497,526

(22) PCT Filed: Dec. 3, 2002
(86) PCT No.: PCT/EP02/13660

§ 371 Date: Jun. 3, 2004

(30) Foreign Application Priority Data
Dec. 4, 2001 (DE) 101 59 369.1

(51) Int. Cl. C12P 25/00; C12N 1/21

(52) U.S. Cl. 435/66; 435/252.3

(57) ABSTRACT

Process for the microbial production of riboflavin by growing a microorganism of the genus Ashbya which is capable of riboflavin production and which shows higher activities than the wild type ATCC 10895 in at least two of the gene products selected from the group consisting of rib1, rib2, rib4 and rib7, and subsequently isolating the produced riboflavin from the culture medium.

The present invention relates to a recombinant process for the production of riboflavin. Owing to the specific selection of riboflavin biosynthesis genes or their combination in organisms of the genus Ashbya and their expression, the production of riboflavin in these organisms is increased.

Vitamin B2, also referred to as riboflavin, is produced by all plants and a multiplicity of microorganisms. It is essential for humans and animals since they are not capable of synthesizing it. Riboflavin plays an important role in the metabolism. Thus, for example, it is involved in carbohydrate utilization. Vitamin B2 deficiency results in inflammations of the oral and pharyngeal mucous membranes, itching and inflammation in cutaneous folds and similar skin damage, conjunctivitis, reduced visual acuity and corneal opacification. In babies and children, inhibition of growth and weight loss may occur. Vitamin B2 is therefore of great economic importance, for example as vitamin supplement in the event of vitamin deficiency and as feed additive. It is also added to a variety of foodstuffs. In addition, it is also used as food coloring, for example in mayonnaise, ice cream, blancmange and the like.

Vitamin B2 is either prepared chemically or produced microbially (see, for example, B. Kurth et al., 1996, Riboflavin, in: Ullmann's Encyclopedia of industrial chemistry, VCH Weinheim). In chemical syntheses, riboflavin is, as a rule, obtained in multi-step processes as pure end product, it being necessary to employ relatively expensive starting materials such as, for example, D-ribose.

An alternative to the chemical synthesis of riboflavin is the fermentation of microorganisms to produce vitamin B2. Starting materials which are used for this purpose are renewable raw materials such as sugars or vegetable oils. The production of riboflavin by fermenting fungi such as Eremothecium ashbyii or Ashbya gossypii is known (The Merck Index, Windholz et al., eds. Merck & Co., page 1183, 1983), but yeasts such as, for example, Candida, Pichia and Saccharomyces or bacteria such as, for example, Bacillus, Clostridium species or Coryne bacteria have also been described as riboflavin producers. EP-A-0 405 370 and EP-A-0 821 063 describe the production of riboflavin with recombinant bacterial strains, the strains having been obtained by transformation of Bacillus subtilis with riboflavin biosynthesis genes.

WO 95/26406 and WO 94/11515 describe how the genes which are specific for riboflavin biosynthesis are cloned from the eukaryotic organisms Ashbya gossypii and Saccharomyces cerevisiae, microorganisms which have been transformed with these genes and the use of such microorganisms for riboflavin synthesis.

WO 99/61623 describes how the choice of riboflavin biosynthesis genes (rib3, rib4, rib5) are used for increasing riboflavin production.

In both of the abovementioned organisms, 6 enzymes catalyze the production of riboflavin starting from guanosine triphosphate (GTP) and ribulose-5-phosphate. GTP cyclohydrolase-II (rib1 gene product) converts GTP into 2,5-diamino-6-(ribosylamino)-4-(3H)-pyrimidinone-5-phosphate. This compound is subsequently reduced by 2,5-diamino-6-(ribosylamino)-4-(3H)-pyrimidinone-5-phosphate reductase (rib7 gene product) to give 2,5-diamino-ribitylamino-2,4-(1H,3H)-pyrimidine-5-phosphate, which is then deaminated by a specific deaminase (rib2 gene product) to give 5-amino-6-ribitylamino-2,4-(1H,3H)-pyrimidinedione-5-phosphate. The phosphate is then eliminated by an unspecific phosphatase.

Ribulose-5-phosphate, besides GTP the second starting material of the last enzymatic steps of riboflavin biosynthesis, is converted by 3,4-dihydroxy-2-butanone-4-phosphate synthase (rib3 gene product) to give 3,4-dihydroxy-2-butanone-4-phosphate (DBP).

Both DBP and 5-amino-6-ribitylamino-2,4-(1H,3H)-pyrimidinedione are the starting materials of the enzymatic synthesis of 6,7-dimethyl-8-ribityllumazine. This reaction is catalyzed by the rib4 gene product (DMRL synthase). DMRL is thereupon converted into riboflavin by riboflavin synthase (rib5 gene product) (Bacher et al. (1993), Bioorg. Chem. Front. Vol. 3, Springer Verlag).

Despite these advances in riboflavin production, there remains a need of improving and increasing the vitamin B2 productivity in order to meet the increasing demand and to make the production of riboflavin more efficient.

It is an object of the present invention to further improve vitamin B2 productivity. We have found that this object is achieved by a process for the microbial production of riboflavin by growing a microorganism of the genus Ashbya which is capable of producing riboflavin and which shows higher ativities than the wild type ATCC 10895 in at least two of the gene products selected from the group consisting of rib1, rib2, rib4 and rib7, and subsequently isolating the riboflavin produced from the culture medium.

The process for the increased production of riboflavin is preferably carried out with an organism which is capable of synthesizing riboflavin and in which for example the combination of the following rib gene products show an increased activity (the numbers indicate in each case the rib gene product in question): 1+2, 1+4, 1+7, 2+4, 2+7, 4+7.

Especially preferred are those organisms in which the combination of the following rib gene products show an increased activity (the numbers indicate in each case the rib gene product in question): 1+2+4, 1+2+7, 1+4+7, 2+4+7.

The increased activity of the rib gene products is assessed in comparison with the Ashbya gossypii strain ATCC 10895 which is used as reference organism. The relevant processes of measuring the activity of the rib gene products, i.e. the enzyme activities, are known to the skilled worker and described in the literature.

Furthermore advantageous for increasing vitamin B2 productivity is the combination of increasing the natural enzyme activity and introducing the abovementioned gene combination for increasing gene expression.

Suitable organisms or host organisms for the process according to the invention are, in principle, all organisms of the genus Ashbya which are capable of synthesizing riboflavin.

The term rib gene products refers not only to the polypeptide sequences described in the sequence listing in SEQ ID NO: 2, 4, 6, 8, but also those polypeptide sequences which can be obtained from these sequences by substitution, insertion or deletion of up to 5%, preferably up to 3%, especially preferably up to 2%, of the amino acid codons. Such sequences occur for example as natural allelic variations or can be obtained by mutagenic treatment of the original strain, for example by mutagenic substances or electromagnetic radiation and subsequent selection for increased riboflavin productivity.

The combination according to the invention of the rib genes rib1, rib2, rib4 and rib7 and/or the increased activity of the genes and their gene products brings about a markedly increased riboflavin productivity. The abovementioned genes can be introduced into the organisms used by, in principle, all methods known to the skilled worker; advantageously, they are introduced into the organisms or their cells by transformation, transfection, electroporation, using what is known as the gene gun, or by microinjection. In the case of microorganisms, the skilled worker will find suitable methods in the textbooks by Sambrook, J. et al. (1989) Molecular cloning: A laboratory manual, Cold Spring Harbor Laboratory Press, by F. M. Ausubel et al. (1994) Current protocols in molecular biology, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Harbor Laboratory Press or Guthrie et al. Guide to Yeast Genetics and Molecular Biology, Methods in Enzymology, 1994, Academic Press. Examples of advantageous methods which may be mentioned are the introduction of DNA by homologous or heterologous recombination, for example with the aid of the the ura-3 gene, specifically the Ashbya ura-3 gene, as described in the German application DE 19801120.2 and/or by the REMI method (=“Restriction Enzyme Mediated Integration”) described hereinbelow.

The REMI technique is based on cotransforming an organism with a linear DNA construct which has been cleaved at both ends with the same restriction endonuclease and this restriction endonuclease which has been used for restricting the DNA construct. Thereupon, the restriction endonuclease cleaves the genomic DNA of the organism into which the DNA construct together with the restriction enzyme has been introduced. This leads to the activation of the homologous repair mechanisms. These repair mechanisms repair the strand breaks of the genomic DNA which have been caused by the endonuclease and, by doing so, also incorporate, at a certain frequency, the cotransformed DNA construct into the genome. As a rule, the restriction cleavage sites at both ends of the DNA are retained.

This technique has been described by Bolker et al. (Mol Gen Genet, 248, 1995: 547-552) for the insertion mutagenesis of fungi. The method was used by Schiestl and Petes (Proc. Natl. Acad. Sci. USA, 88, 1991: 7585-7589) for elucidating the existence of heterologous recombination in Saccharomyces. The method has been described by Brown et al. (Mol. Gen. Genet. 251, 1996: 75-80) for the stable transformation and regulated expression of an inducible reporter gene. As yet, the system has not been used as a tool of genetic engineering for optimizing etabolic pathways or for the commercial overexpression of proteins.

It has been shown with reference to riboflavin synthesis that biosynthesis genes can be integrated into the genome of the abovementioned organisms with the aid of the REMI method and that production processes for producing metabolites of the primary or secondary metabolism can be optimized, specifically of biosynthetic pathways, for example of amino acids such as lysine, methionine, threonine or tryptophan, vitamins such as vitamins A, B2, B6, B12, C, D, E, F, S-adenosylmethionine, biotin, pantothenic acid or folic acid, carotenoids such as β-carotene, lycopin, canthaxanthin, astaxanthin or zeaxanthin, or proteins such as hydrolases like lipases, esterases, amidases, nitrilases, proteases, mediators such as cytokins, for example lymphokins such as MIF, MAF, TNF, interleukins such as interleukin 1, interferones such as γ-interferone, tPA, hormones such as proteohormones, glycohormones, oligo- or polypeptide hormones such as vasopressin, endorphins, endostatin, angiostatin, growth factors erythropoietin, transcription factors integrins such as GPIIb/IIIa or αᵥβIII, receptors such as the various glutamate receptors or angiogenesis factors such as angiotensin.

Using the REMI method, the nucleic acid fragments according to the invention or other of the abovementioned genes can be placed at transcription-active sites in the genome.

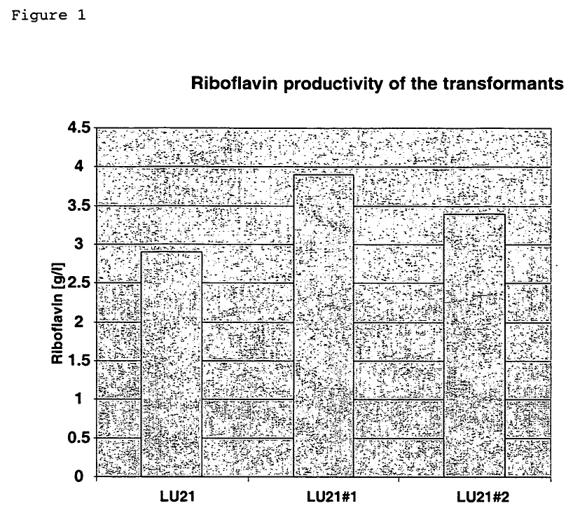

It is advantageous to clone the nucleic acid together with at least one reporter gene into a DNA construct which is introduced into the genome. This reporter gene should make possible an easy detectability via a growth assay, fluorescence assay, chemoluminescence assay, bioluminescence assay or via photometrical measurement. Examples of reporter genes which may be mentioned are genes for resistance to antibiotics, hydrolase genes, fluorescence protein genes, bioluminescence genes, glucosidase genes, peroxidase genes or biosynthesis genes such as the riboflavin genes, the luciferase gene, β-galactosidase gene, gfp gene, lipase gene, esterase gene, peroxidase gene, β-lactamase gene, acetyltransferase gene, phosphotransferase gene or adenyl transferase gene. These genes make possible the easy measurement and quantification of the transcriptional activity and thus of gene expression. Thus, locations in the genome can be identified which show a productivity which differs by up to a factor of 2 (see FIG. 1). FIG. 1 shows clones Lu22#1 and LU21#2, which were obtained after integration, together with their different vitamin B2 (=riboflavin) productivities.

In the event that the biosynthesis genes themselves make possible an easy detectability, as is the case, for example, with riboflavin, an additional reporter gene can be dispensed with.

If more than one gene is to be introduced into the organism all of these can be introduced into the organism together with a reporter gene in a single vector, or each individual gene can be introduced into the organism together with a reporter gene in one respective vector, it being possible to introduce the various vectors simultaneously or successively. Gene fragments which encode for the activity in question may also be employed in the REMI technique.

In principle, all of the known restriction enzymes are suitable for the process according to the invention for the integration of biosynthesis genes into the genome of organisms. Restriction enzymes which recognize only 4 base pairs as restriction cleavage sites are less preferred since they cleave too frequently within the genome or the vector to be integrated; preferred are enzymes which recognize 6, 7, 8 or more base pairs as cleavage sites, such as BamHI, EcoRI, BglII, SphI, SpeI, XbaI, XhoI, NcoI, SalI, ClaI, KpnI, HindIII, SacI, PstI, BpnI, NotI, SrfI or SfiI, to mention but some of the possible enzymes. It is advantageous when the enzymes used no longer have cleavage sites in the DNA to be introduced; this increases the integration efficacy. As a rule, 5 to 500 U, preferably 10 to 250 U, especially preferably 10 to 100 U of the enzymes are used in the REMI mix. The enzymes are advantageously employed in an aqueous solution comprising substances for osmotic stabilization such as sugars like sucrose, trehalose or glucose, polyols such as glycerol or polyethylene glycol, a buffer with an advantageous buffer range of from pH 5 to 9, preferably 6 to 8, especially preferably 7 to 8, such as Tris, MOPS, HEPES, MES or PIPES and/or substances for stabilizing the nucleic acids, such as inorganic or organic salts of Mg, Cu, Co, Fe, Mn or Mo. If appropriate, further substances such as EDTA, EDDA, DTT, β-mercaptoethanol or nuclease inhibitors may furthermore be present. However, it is also possible to carry out the REMI technique without these additions.

The process according to the invention is carried out in a temperature range of from 5 to 80° C., preferably 10 to 60° C., especially preferably 20 to 40° C. All the known methods for destabilizing cell membranes are suitable for the process, such as, for example, electroporation, fusion with loaded vesicles or destabilization by means of various alkali metal salts or alkaline earth metal salts such as lithium salts, rubidium salts or calcium salts, with the lithium salts being preferred.

After the isolation, the nucleic acids can be used for the reaction according to the invention either directly or following purification.

The introduction into plants of the combination according to the invention of the rib genes can be effected in principle by all the methods known to the skilled worker.

The transfer of foreign genes into the genome of the plant is termed transformation. In this context, the described methods of transforming and regenerating plants from plant tissues or plant cells are used for the transient or stable transformation. Suitable methods are the protoplast transformation by polyethylene glycol-induced DNA uptake, the use of a gene gun, electroporation, the incubation of dry embryos in DNA-containing solution, microinjection and the Agrobacterium-mediated gene transfer. The abovementioned methods are described for example in B. Jenes et al., Techniques for Gene Transfer, in: Transgenic Plants, Vol. 1, Engineering and Utilization, edited by S. D. Kung and R. Wu, Academic Press (1993) 128-143 and in Potrykus Annu. Rev. Plant Physiol. Plant Molec. Biol. 42 (1991) 205-225. Preferably, the construct to be expressed is cloned into a vector which is suitable for transforming Agrobacterium tumefaciens, for example pBin19 (Bevan et al., Nucl. Acids Res. 12 (1984) 8711). The transformation of plants with Agrobacterium tumefaciens is described, for example, by Höfgen und Willmitzer in Nucl. Acid Res. (1988) 16, 9877.

Agrobacteria transformed with an expression vector according to the invention can also be used in the known manner for transforming plants, in particular crop plants, such as cereals, maize, soybean, rice, cotton, sugarbeet, canola, sunflower, flax, hemp, potato, tobacco, tomato, oilseed rape, alfalfa, lettuce and the various tree, nut and grapevine species, and also legumes, for example by bathing scarified leaves or leaf sections in an agrobacterial solution and subsequently growing them in suitable media.

The genetically modified plant cells can be regenerated by all methods known to the skilled worker. Suitable methods can be found in the abovementioned publications by S. D. Kung and R. Wu, Potrykus or Höfgen and Willmitzer.

There exists a multiplicity of possibilities of increasing the enzyme activity of the rib gene products in the cell.

One possibility consists in modifying the endogenous rib genes 1, 2, 4 and 7 in such a way that they encode for enzymes whose 1, 2, 4 or 7 activity is increased over that of the starting enzymes. A different increase in the enzyme activity can be achieved for example by bringing about an increased substrate conversion owing to a change in the catalytic centers, or by neutralizing the effect of enzyme inhibitors, that is to say they have an increased specific activity, or their activity is not inhibited. In a further advantageous embodiment, an increased enzyme activity may also be effected by increasing enzyme synthesis in the cell, for example by eliminating factors which repress enzyme synthesis or by increasing the activity of factors or regulatory elements which promote increased synthesis, or, preferably, by introducing further gene copies. These measures increase the total activity of the gene products in the cell without modifying the specific activity. A combination of these methods may also be used, that is to say both the specific activity and the total activity are increased. In principle, these modifications can be introduced into the nucleic acid sequences of the genes, regulatory elements or their promoters by all methods known to the skilled worker. To this end, the sequences can, for example, be subjected to a mutagenesis such as a site-directed mutagenesis as is described in D. M. Glover et al., DNA Cloning Vol. 1, (1995), IRL Press (ISBN 019-963476-9), chapter 6, page 193 et seq.

A PCR method for random mutagenesis using dITP is described by Spee et al. (Nucleic Acids Research, Vol. 21, No. 3, 1993: 777-778).

The use of an in-vitro recombinant technique for molecular evolution is described by Stemmer (Proc. Natl. Acad. Sci. USA, Vol. 91, 1994: 10747-10751).

Moore et al. (Nature Biotechnology Vol. 14, 1996: 458-467) describes the combination of the PCR method and the recombinant method.

The modified nucleic acid sequences are subsequently returned into the organisms via vectors.

To increase the enzyme activities, it is also possible to place modified promoter regions upstream of the natural genes so that the expression of the genes is enhanced and, eventually, the activity increased. It is also possible to introduce, at the 3′ end, sequences which increase for example the stability of the mRNA and thus bring about an increased translation. This also leads to increased enzyme activity.

Preferably, further gene copies of the rib genes 1, 2, 7 and 4 are introduced jointly into the cell. These gene copies can be subject to the natural regulation, to a modified regulation where the natural regulatory regions have been modified in such a way that they make possible an increased expression of the genes, or else regulatory sequences of heterologous genes or indeed of genes from different species may be used.

A combination of the abovementioned methods is especially advantageous.

The combination of the genes of sequences SEQ ID No. 1, SEQ ID No. 3, SEQ ID No. 5 and SEQ ID No. 7 or their functional equivalents is advantageous in the process according to the invention.

To bring about optimal expression of heterologous genes in organisms, it is advantageous to modify the nucleic acid sequences in accordance with the specific codon usage of the organism. The codon usage can be determined readily by computer evaluations of other, known genes of the organism in question.

The gene expression of the rib genes 1, 2, 7 and 4 can be increased advantageously by increasing the rib 1, 2, 7, 4 gene copy number and/or by enhancing regulatory factors which have a positive effect on rib1, 2, 7 and 4 gene expression. Thus, an enhancement of regulatory elements can preferably be effected at the transcriptional level by using stronger transcription signals such as promoters and enhancers. Besides, however, an enhancement of translation is also possible, for example by improving the stability of the rib1, 2, 7 and 4 mRNA or by increasing the reading efficacy of this mRNA at the ribosomes.

To increase the gene copy number, it is possible to incorporate the rib genes 1, 2, 7 and 4, or homologous genes for example into a nucleic acid fragment or into a vector which preferably comprises the regulatory gene sequences assigned to the rib genes in question or a promoter activity with analogous effect.

Regulatory sequences which are used in particular are those which increase gene expression. As an alternative, however, each of the above-described genes can be introduced into a separate vector and transformed into the production organism in question.

The nucleic acid fragment according to the invention is understood as meaning the rib gene sequences SEQ ID No. 1, SEQ ID No. 3, SEQ ID No. 5 and SEQ ID No. 7 or their functional equivalents which were linked operably with one or more regulatory signals, preferably for increasing gene expression. These regulatory sequences are, for example, sequences to which inductors or repressors bind and thus regulate the expression of the nucleic acid. In addition to these novel regulatory sequences, or instead of these sequences, the natural regulation of these sequences may still be present upstream of the actual structural genes and, if appropriate, may have been genetically modified in such a way that the natural regulation was eliminated and expression of the genes increased. However, the gene construct may also be simpler in structure, that is to say no additional regulatory signals were inserted upstream of the sequences SEQ ID No. 1, SEQ ID No. 3, SEQ ID No. 5 or SEQ ID No. 7 or their functional equivalents and the natural promoter together with its regulation was not removed. Instead, the natural regulatory sequence was mutated in such a way that regulation no longer takes place and gene expression is increased. These modified promoters may also be inserted on their own upstream of the natural genes in order to increase the activity. Moreover, the gene construct may advantageously also comprise one or more of what are known as enhancer sequences in operable linkage with the promoter, and these make possible an increased expression of the nucleic acid sequence. Also, additional advantageous sequences such as further regulatory elements or terminators may be inserted at the 3′ end of the DNA sequences. One or more copies of the rib genes may be present in the gene construct.

Examples of advantageous regulatory sequences for the process according to the invention are present for example in promoters such as cos, tac, trp, tet, trp-tet, lpp, lac, lpp-lac, lacI^{q}, T7, T5, T3, gal, trc, ara, SP6, λ-P_{R} or in the λ-P_{L} promoter, which are advantageously used in Gram-negative bacteria. Further advantageous regulatory sequences are present for example in the Gram-positive promoters amy and SPO2, in the yeast or fungal promoters ADC1, MFα, AC, P-60, CYC1, GAPDH, TEF, rp28, ADH or in the plant promoters CaMV 35S [Franck et al., Cell 21 (1980) 285-294], PRP1 [Ward et al., Plant. Mol. Biol. 22 (1993)], SSU, OCS, lib4, usp, STLS1, B33, LEB4, nos or in the ubiquitin or phaseolin promoter. In this context, the pyruvate decarboxylase and methanol oxidase promoters from, for example, Hansenula are also advantageous. Further advantageous plant promoters are, for example, a benzenesulfonamide-inducible promoter (EP 388186), a tetracyclin-inducible promoter (Gatz et al., (1992) Plant J. 2, 397-404), an abscisic-acid-inducible promoter (EP 335528) or an ethanol- or cyclohexanone-inducible promoter (WO 9321334). Those plant promoters which ensure the expression in tissues or plant parts in which the biosynthesis of purines or their precursors takes place are particularly advantageous. Promoters which ensure leaf-specific expression must be mentioned in particular. The potato cytosolic FBPase promoter or the potato ST-LSI promoter (Stockhaus et al., EMBO J. 8 (1989) 2445-245) must be mentioned. The Glycine max phosphoribosyl-pyrophosphate amidotransferase promoter (see also Genbank Accession Number U87999) or another nodule-specific promoter as described in EP 249676 may also be used advantageously.

In principle, all natural promoters together with their regulatory sequences, such as those mentioned above, can be used for the process according to the invention. In addition, synthetic promoters may also be used advantageously.

Further genes to be introduced into the organisms may additionally be present in the nucleic acid fragment (=gene construct) as described above. These genes can be subject to separate regulation or else under the same regulatory region as the rib genes. These genes are, for example, further biosynthesis genes which make possible an increased synthesis.

For expression in the abovementioned host organism, the nucleic acid fragment is advantageously inserted into a vector such as, for example, a plasmid, a phage or other DNA, which makes possible optimal expression of the genes in the host. Examples of suitable plasmids are, for example, pLG338, pACYC184, pBR322, pUC18, pUC19, pKC30, pRep4, pHS1, pHS2, pPLc236, pMBL24, pLG200, pUR290, pIN-III¹¹³-B1, λgt11 or pBdCI in E. coli, pIJ101, pIJ364, pIJ702 or pIJ361 in Streptomyces, pUB110, pC194 or pBD214 in Bacillus, pSA77 or pAJ667 in Corynebacterium, pALS1, pIL2 or pBB116 in fungi, 2 μM, pAG-1, YEp6, YEp13 or pEMBLYe23 in yeasts, pLGV23, pGHlac⁺, pBIN19, pAK2004 or pDH51 in plants, or derivatives of the abovementioned plasmids. The abovementioned plasmids constitute a small selection of the plasmids which are possible. Further plasmids are well known to the skilled worker and can be found for example in the book Cloning Vectors (Eds. Pouwels P. H. et al. Elsevier, Amsterdam-New York-Oxford, 1985, ISBN 0 444 904018). Suitable plant vectors are described, inter alia, in “Methods in Plant Molecular Biology and Biotechnology” (CRC Press), chapter 6/7, pp. 71-119.

To express the further genes which are present, the nucleic acid fragment advantageously additionally also comprises 3′- and/or 5′-terminal regulatory sequences for enhancing the expression, which sequences are selected for optimal expression as a function of the chosen host organism and the gene(s).

These regulatory sequences are intended to make possible the directed expression of the genes and of protein expression. Depending on the host organism, this may mean, for example, that the gene is expressed and/or overexpressed only after induction, or that it is immediately expressed and/or overexpressed.

In this context, the regulatory sequences or factors can preferably have a positive effect on, and thus increase, the gene expression of the genes introduced. Thus, an enhancement of the regulatory elements can preferably be effected at the transcriptional level by using strong transcriptional signals such as promoters and/or enhancers. Besides, however, an enchancement of translation is also possible, for example by increasing the stability of the mRNA.

In a further embodiment of the vector, the gene construct according to the invention can advantageously also be introduced into the microorganisms in the form of a linear DNA and integrated into the genome of the host organism via heterologous or homologous recombination. This linear DNA can consist of a linearized plasmid or else only of the nucleic acid fragment as vector.

Any plasmid (but in particular a plasmid which carries the replication origin of the S. cerevisiae 2∝m plasmid) which replicates autonomously in the cell may also be used as vector, but also, as described above, a linear DNA fragment which integrates into the host's genome. This integration can be effected via heterologous or homologous recombination, but preferably, as mentioned, via homologous recombination (Steiner et al., Genetics, Vol. 140, 1995: 973-987). In this context, the genes rib1, rib2, rib4 and rib7 may be present individually in the genome at different locations or on different vectors or else jointly in the genome or on a vector.

The organisms used in the process according to the invention which comprise the combination of the rib genes 1, 2, 7 and 4 or their functional equivalents show an increased riboflavin production.

In the process according to the invention, the organisms used for the production of riboflavin are grown in a medium which allows the growth of these organisms. This medium may take the form of a synthetic or a natural medium. Media known to the skilled worker are used and chosen to suit the organism. The media used comprise a carbon source, a nitrogen source, inorganic salts and, if appropriate, minor amounts of vitamins and trace elements to support the growth of the microorganisms.

Examples of advantageous carbon sources are sugars such as mono-, di- or polysaccharides such as glucose, fructose, mannose, xylose, galactose, ribose, sorbose, ribulose, lactose, maltose, sucrose, raffinose, starch or cellulose, complex sources of sugars such as molasses, sugar phosphates such as fructose-1,6-bisphosphate, sugar alcohols such as mannitol, polyols such as glycerol, alcohols such as methanol or ethanol, carboxylic acids such as citric acid, lactic acid, or acetic acid, fats such as soya oil or rapeseed oil, amino acids such as an amino acid mixture, for example what are known as casamino acids (Difco) or individual amino acids such as glycin or aspartic acid or amino sugars; the last-mentioned may also be used simultaneously as nitrogen source.

Advantageous nitrogen sources are organic or inorganic nitrogen 25 compounds or materials comprising these compounds. Examples are ammonium salts such as NH₄Cl or (NH₄)₂SO₄, nitrates, urea, or complex nitrogen sources such as cornsteep liquor, brewer's yeast autolyzate, soybean meal, wheat gluten, yeast extract, meat extract, casein hydrolyzate, yeast or potato protein, all of which can frequently also simultaneously act as nitrogen source.

Examples of inorganic salts are the calcium, magnesium, sodium, cobalt, molybdenum, manganese, potassium, zinc, copper and iron salts. As anion of these salts, the chloride, sulfate and phosphate ions may be mentioned in particular. An important factor for increasing the productivity in the process according to the invention is the control of the Fe²⁺ or Fe³⁺ ion concentration in the production medium.

If appropriate, the nutrient medium is supplemented with further growth factors such as, for example, vitamins or growth promoters such as biotin, riboflavin, thiamine, folic acid, nicotinic acid, pantothenate or pyridoxine, amino acids such as alanine, cysteine, proline, aspartic acid, glutamine, serine, phenylalanine, ornithine or valine, carboxylic acids such as citric acid, formic acid, pimelic acid or lactic acid, or substances such as dithiothreitol.

The mixing ratio of the abovementioned nutrients depends on the type of fermentation and is adjusted for each individual case. All the components of the medium may be provided at the beginning of the fermentation, if appropriate after having been sterilized separately or jointly, or else they may be fed continuously or batchwise during the fermentation, as required.

The growth conditions are set in such a way that growth of the organisms is optimal and that the best possible yields are achieved. Preferred growth temperatures are at from 15° C. to 40° C. Temperatures of between 25° C. and 37° C. are especially advantageous. The pH value is preferably set within a range of from 3 to 9. pH values of between 5 and 8 are especially advantageous. In general, an incubation time ranging from a few hours to some days, preferably from 8 hours up to 21 days, especially from 4 hours to 14 days, is generally sufficient. The maximum amount of product accumulates in the medium within this period.

The advantageous optimization of media can be found by the skilled worker for example in the textbook Applied Microbial Physiology, “A Practical Approach” (Eds. P. M. Rhodes, P. F. Stanbury, IRL Press, 1997, pages 53-73, ISBN 0 19 963577 3). Advantageous media and growth conditions for Bacillus and further organisms can be found for example in the publication EP-A-0 405 370, in particular Example 9, for Candida in the publication WO 88/09822, in particular Table 3, and for Ashbya in the publication by Schmidt et al. (Microbiology, 142, 1996: 30 419-426).

The process according to the invention can be carried out continuously or discontinuously as a batch culture or fed-batch culture.

Depending on the level of the initial productivity of the organism used, the riboflavin productivity can be increased less or more by the process according to the invention. As a rule, the productivity can be increased advantageously by at least 5%, preferably by at least 10%, especially preferably by 20%, very especially preferably by at least 100% over that of the starting organism.

### EXAMPLES

The isolation of the rib genes 1, 2, 3, 4, 5 and 7 from Ashbya gossypii and Saccharomyces cerevisiae is described in Patents WO 95/26406 and WO 93/03183 and specifically in the examples and was carried out analogously. These publications are herewith expressly referred to.

Sequence 1 shows the DNA construct which, besides the selection marker required for transformation, carries the rib1, rib2, rib4 and rib7 gene fragments.

General methods such as, for example, cloning, restriction cleavages, agarose gel electrophoresis, linkage of DNA fragments, transformation of microorganisms, growing bacteria and the sequence analysis recombinant DNA were carried out as described by Sambrook et al. (1989) (Cold Spring Harbor Laboratory Press: ISBN 0-87969-309-6), unless otherwise specified.

Recombinant DNA molecules were sequenced using an ABI laser fluorescence DNA sequencer, following the method of Sanger (Sanger et al. (1977) Proc. Natl. Acad. Sci. USA74, 5463-5467). Fragments resulting from a polymerase chain reaction were sequenced and verified to avoid polymerase errors in constructs to be expressed.

### Example 1

Cloning of the DNA construct comprising the rib1, rib2, rib4 and rib7 gene copies (vector Tef-G418-Tef rib1,2,7,4)

Expression constructs of the rib genes: the vector TefG418Tefrib3,4,5 is described in WO 99/61623. This vector was cut with KpnI, precipitated, redissolved and subsequently partially digested with NheI. The larger fragment which had been cut once with NheI and KpnI has been isolated from an agarose gel. The rib7 gene was amplified from vector pJR765 (described in WO 95/26406) with the aid of PCR (primer: TCGAGGTACCGGGCCCCCCCTCGA; TCGAACTAGTAGACCAGTCAT). The specific PCR product was cut with KpnI/SpeI and ligated with the above-described KpnI/NheI-cut vector. This gave rise to vector TefG418Tefrib7,4.

The rib2 gene has been amplified from vector pJR758 (WO 95/26406) by PCR, and the resulting product has been cut with SpeI and NheI (primer: CCCAACTAGTCTGCAGGACAATTTAAA; AGTGCTAGCCTACAATTCGCAGCAAAAT). This DNA fragment has been ligated with the NheI-cut, phosphatase-treated vector TefG418Tefrib7,4. This gave rise to vector TefG418Tefrib7,4,2. The rib1 gene has been amplified from vector pJR765 (WO 95/26406) by PCR (primer: GTAGTCTAGAACTAGCTCGAAACGTG; GATTCTAGAACTAGAACTAGTGGATCCG) and was cut with XbaI. This DNA fragment has been ligated with the NheI-cut, phosphatase-treated vector TefG418Tefrib7,4,2.

The resulting DNA construct constitutes the vector Tef-G418-rib1,2,7,4.

### Example 2

Transformation of the DNA construct into the fungus Ashbya gossypii.

The DNA construct described in Example 1 (vector Tef-G418-rib1,2,4,7) was cut completely with the restriction enzyme XbaI and the insert which carries the rib gene sequences was purified by agarose gel separation.

MA2 medium (10 g/l Bacto peptone, 1 g/l yeast extract, 0.3 g/l myo-inositol and 10 g/l D-glucose) was inoculated with Ashbya gossypii spores. The culture was incubated for 12 hours at 4° C. and subsequently with shaking for 13 hours at 28° C. The cell suspension was spun down and the cell pellet was taken up in 5 ml 50 mM potassium phosphate buffer pH 7.5, 25 mM DTT. After heat treatment for 30 minutes at 28° C., the cells were again spun down and taken up in 25 ml of STM buffer (270 mM sucrose, 10 mM TRIS-HC1 pH 7.5, 1 MM MgCl₂). 0.5 ml of this suspension was then treated with approx. 3 μg of the above purified insert and 40 U SpeI enzyme and electroporated in a Biorad Gene Pulser (100 Ω, 20 μF, 1.5 kV). After the electroporation, the cells have been treated with 1 ml of MA2 medium and plated onto MA2 agar culture plates. To perform the selection with antibiotics, the plates were incubated for 5 hours at 28° C. and then covered with a layer of 5 ml low-melting agarose comprising the antibiotic G418 (200 μg/ml). The transformants were subjected to clonal purification by micromanipulation (Steiner and Philipsen (1995) Genetics, 140; 973-987). The successful integration of the construct was verified by subjecting the genomic DNA transformants to PCR analysis. The genomic DNA was isolated as described by Carle and Olson (Proc. Natl. Acad. Sci, 1985, 82, 3756-3760) and Wright and Philipsen (Gene, 1991, 109, 99-105). The PCR was carried out with construct-specific primers by the method of R. Saiki (PCR Protocols, 1990, Academic Press, 13-20). The PCR fragments are analyzed by separation in an agarose gel

A successful integration into the genome was confirmed for all transformants by means of PCR.

### Example 3

Riboflavin determination in the recombinant Ashbya gossypii clone.

Ashbya gossypii LU21 (wild-type strain, ATCC 10895) and the strains LU21#1 and #2 obtained therefrom by transformation with the construct described in Example 1 were grown for 4 days on agar medium at 28° C. Three 100 ml Erlenmeyer flasks containing 10 ml of medium (27.5 g/l yeast extract, 0.5 g/l MgSO₄, 50 ml/l soy oil, pH 7.0) were inoculated from this plate. After incubation on the shaker for 40 hours at 28° C. and 180 rpm, 1 ml portions of the culture liquid were transferred into 250 ml Erlenmeyer flasks containing 20 ml of YPD medium (10 g/l yeast extract, 20 g/l Bacto-peptone, 20 g/l glucose). Incubation at 28° C. and 300 rpm. After 190 hours, a 1 ml sample was taken from each flask and treated with 1 ml of 1 M perchloric acid. The sample was filtered, and the riboflavin content was determined by HPLC analysis. A calibration with riboflavin standards (10 mg/l, 20 mg/l, 30 mg/l, 40 mg/l, 50 mg/l) was carried out.

Parameters of the HPLC method for determining riboflavin:

| | |
|---|---|
| Column | ODS Hypersil 5 mm 200 × 2.1 mm (HP) |
| Eluent A | water with 340 ml H₃PO₄ (89%) to pH 2.3 |
| Eluent B | 100% acetonitrile |
| Gradient | |
| Stop time | 0 to 6 min.: 2% B to 50% B |
| | 6 to 6.5 min: 50% B to 2% B |
| Flow rate | 0.5 ml/min |
| Detection | 280 nm |
| Temperature | 40° C. |
| Injection | 2 to 10 μl |

In comparison with the initial strain, the batches with clones #1 and #2, which contain an additional gene copy of the rib genes 1, 2, 4 and 7 show a markedly increased riboflavin productivity (FIG. 1).

FIG. 1 shows the riboflavin yields of the different clones. Increases in the riboflavin yields of up to 135% in comparison with the unmodified strain were obtained by introducing the rib1, 2, 4 and 7 genes.

1. A process for microbial production of riboflavin by comprising:
growing in a culture medium a microorganism of the genus Ashbya which is capable of producing riboflavin that shows a higher riboflavin activity for at least two riboflavin biosynthesis gene products than a microorganism of ATCC 10895 in wherein the at least two riboflavin biosynthesis gene products are selected from the group consisting of rib1, rib2, rib4 and rib7; and
isolating the riboflavin from the culture medium.

2. The process of claim 1, wherein the at least two gene products comprise three gene products.

3. The process of claim 1, wherein the at least two gene products comprise four gene products.

4. The process of claim 1, wherein the higher riboflavin activity of the at least two gene products is brought about by an increased gene expression.

5. The process of claim 4, wherein the increased gene expression is brought about by an increased gene copy number.

6. The process of claim 1, wherein one of the at least two gene products comprises the polypeptide sequence of SEQ ID NO: 2, 4, 6, 8 or another polypeptide sequence that can be obtained from the polypeptide sequence of SEQ ID NO: 2, 4. 6, or 8 after substitution, insertion, deletion or a combination thereof of up to 5% of the amino acid codons present within said polypeptide sequence.

7. The process of claim 1, wherein the at least two gene products comprises rib1 and rib2, rib1 and rib4, rib1 and rib7, rib2 and rib4, rib2 and rib7 or rib4 and rib7.

8. The process of claim 2, wherein the three gene products comprises rib1, rib2 and rib4; rib1, rib2 and rib7; rib1, rib4 and rib7; or rib2, rib4 and rib7.

9. The process of claim 6, wherein the another polypeptide sequence contains a substitution, insertion, deletion or combination thereof of up to 3% of the amino acid codons present in said polypeptide sequence.

10. The process of claim 6, wherein the another polypeptide sequence contains a substitution, insertion, deletion or combination thereof of up to 2% of the amino acid codons present in said polypeptide sequence.

11. The process of claim 1, wherein the higher riboflavin activity of the at least two gene products is brought about by introducing one or more additional genes to said microorganism which may by derived from the same or a different species.

12. The process of claim 1, wherein the higher riboflavin activity of the at least two gene products is brought about by increasing enzyme synthesis of said microorganism.

13. The process of claim 12, wherein the increased enzyme synthesis is brought about by neutralizing an inhibitor, eliminating a factor that represses enzyme synthesis, increasing activity of a factor that promotes enzyme synthesis, introducing a regulatory sequence into said microorganism, placing a promoter upstream of a gene whose expression increases enzyme synthesis, or a combination thereof.

14. The process of claim 1, wherein the higher riboflavin activity is 5% higher.

15. The process of claim 1, wherein the higher riboflavin activity is 10% higher.

16. The process of claim 1, wherein the higher riboflavin activity is 20% higher.

17. The process of claim 1, wherein the higher riboflavin activity is 100% higher.

18. The process of claim 1, wherein microbial production of riboflavin is inducible.

19. Riboflavin produced by the process of claim 1.

20. A microorganism of the genus Ashbya that is capable of producing at least 110% of the riboflavin of another microorganism of ATCC 10895.

21. The microorganism of claim 20, wherein the microorganism produces at least 150% of the riboflavin of said another microorganism of ATCC 10895.

22. The microorganism of claim 20, wherein production of riboflavin is measured by activity or quantity of riboflavin produced.

23. A method for microbial production of riboflavin comprising growing a microorganism of the genus Ashbya that is capable of producing riboflavin wherein the riboflavin produced therefrom has an activity of at least 110% of the riboflavin activity produced by another microorganism of ATCC 10895 as measured for at least two riboflavin biosynthesis gene products.

24. The method of claim 23, wherein the at least two riboflavin biosynthesis gene products are selected from the group consisting of rib1, rib2, rib4 and rib7.

25. Riboflavin produced by the method of claim 23.
